(19) United States
(12) Patent Application Publication
(10) Pub. No.: US 2007/0293531 A1
(43) Pub. Date: Dec. 20, 2007

(54) Muscarinic Acetycholine Receptor Antagonists

(76) Inventors: Jakob Busch-Petersen, King of Prussia, PA (US); Roderick S. Davis, King of Prussia, PA (US); Dramane Ibrahim Laine, King of Prussia, PA (US); Christopher E. Neipp, King of Prussia, PA (US); Michael R. Palovich, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/573,097

(22) PCT Filed: Aug. 5, 2005
(86) PCT No.: PCT/US05/27958

§ 371 Date: Feb. 2, 2007

Related U.S. Application Data
(60) Provisional application No. 60/600,538, filed on Aug. 5, 2004.

(51) Int. Cl. A61K 31/439; C07D 221/22

(52) U.S. Cl. 514/299; 546/112

(57) ABSTRACT

Muscarinic Acetylcholine Receptor Antagonists and methods of using them are provided.

### FIELD OF THE INVENTION

This invention relates to a series of 9-azabicyclo[3.3.1]nonane derivatives, pharmaceutical compositions, and use thereof in treating muscarinic acetylcholine receptor mediated diseases of the respiratory tract.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs where they mediate many of the vital functions. Muscarinic receptors can mediate both inhibitory and excitatory actions. For example, in smooth muscle found in the airways, M₃ mAChRs mediate contractile responses. For review, please see Caulfield (1993 Pharmac. Ther. 58:319-79).

In the lungs, mAChRs have been localized to smooth muscle in the trachea and bronchi, the submucosal glands, and the parasympathetic ganglia. Muscarinic receptor density is greatest in parasympathetic ganglia and then decreases in density from the submucosal glands to tracheal and then bronchial smooth muscle. Muscarinic receptors are nearly absent from the alveoli. For review of mAChR expression and function in the lungs, please see Fryer and Jacoby (1998 Am J Respir Crit Care Med 158(5, pt 3) S 154-60).

Three subtypes of mAChRs have been identified as important in the lungs, M₁, M₂ and M₃ mAChRs. The M₃ mAChRs, located on airway smooth muscle, mediate muscle contraction. Stimulation of M₃ mAChRs activates the enzyme phospholipase C via binding of the stimulatory G protein Gq/11 (Gs), leading to liberation of phosphatidyl inositol-4,5-bisphosphate, resulting in phosphorylation of contractile proteins. M₃ mAChRs are also found on pulmonary submucosal glands. Stimulation of this population of M₃ mAChRs results in mucus secretion.

M₂ mAChRs make up approximately 50-80% of the cholinergic receptor population on airway smooth muscles. Although the precise function is still unknown, they inhibit catecholaminergic relaxation of airway smooth muscle via inhibition of cAMP generation. Neuronal M₂ mAChRs are located on postganglionic parasympathetic nerves. Under normal physiologic conditions, neuronal M₂ mAChRs provide tight control of acetylcholine release from parasympathetic nerves. Inhibitory M₂ mAChRs have also been demonstrated on sympathetic nerves in the lungs of some species. These receptors inhibit release of noradrenaline, thus decreasing sympathetic input to the lungs.

M₁ mAChRs are found in the pulmonary parasympathetic ganglia where they function to enhance neurotransmission. These receptors have also been localized to the peripheral lung parenchyma, however their function in the parenchyma is unknown.

Muscarinic acetylcholine receptor dysfunction in the lungs has been noted in a variety of different pathophysiological states. In particular, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation (Fryer et al. 1999 Life Sci 64 (6-7) 449-55). This mAChR dysfunction results in airway hyperreactivity and hyperresponsiveness mediated by increased stimulation of M₃ mAChRs. Thus the identification of potent mAChR antagonists would be useful as therapeutics in these mAChR-mediated disease states.

COPD is an imprecise term that encompasses a variety of progressive health problems including chronic bronchitis, chronic bronchiolitis and emphysema, and it is a major cause of mortality and morbidity in the world. Smoking is the major risk factor for the development of COPD; nearly 50 million people in the U.S. alone smoke cigarettes, and an estimated 3,000 people take up the habit daily. As a result, COPD is expected to rank among the top five as a world-wide health burden by the year 2020. Inhaled anti-cholinergic therapy is currently considered the “gold standard” as first line therapy for COPD (Pauwels et al. 2001 Am. J. Respir. Crit. Care Med. 163:1256-1276).

Despite the large body of evidence supporting the use of anti-cholinergic therapy for the treatment of airway hyperreactive diseases, relatively few anti-cholinergic compounds are available for use in the clinic for pulmonary indications. More specifically, in United States, Ipratropium Bromide (Atrovent©; and Combivent©, in combination with albuterol) is currently the only inhaled anti-cholinergic marketed for the treatment of airway hyperreactive diseases. While this compound is a potent anti-muscarinic agent, it is short acting, and thus must be administered as many as four times daily in order to provide relief for the COPD patient. In Europe and Asia, the long-acting anti-cholinergic Tiotropium Bromide (Spiriva©) was recently approved, however this product is currently not available in the United States. Thus, there remains a need for novel compounds that are capable of causing blockade at mAChRs which are long acting and can be administered once-daily for the treatment of airway hyperreactive diseases such as asthma and COPD.

Since mAChRs are widely distributed throughout the body, the ability to apply anti-cholinergics locally and/or topically to the respiratory tract is particularly advantageous, as it would allow for lower doses of the drug to be utilized. Furthermore, the ability to design topically active drugs that have long duration of action, and in particular, are retained either at the receptor or by the lung, would allow the avoidance of unwanted side effects that may be seen with systemic anti-cholinergic use.

### SUMMARY OF THE INVENTION

This invention provides for a method of treating a muscarinic acetylcholine receptor (mAChR) mediated disease, wherein acetylcholine binds to an mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

The compounds according to this invention have the structure shown by Formula (I):

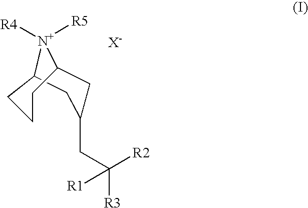

wherein:

the orientation of the alkyl chain attached to the tropane ring is either exo or endo;

R1 is, independently, OH, CN or hydrogen;

R2 and R3 are, independently, selected from the group consisting of straight or branched chain lower alkyl groups, having preferably from 1 to 6 carbon atoms, cycloalkyl groups, having from 5 to 6 carbon atoms, cycloalkyl-alkyl, having 6 to 10 carbon atoms, 2-thienyl, optionally substituted 2-thienyl, 3-thienyl, optionally substituted 3-thienyl, 2-pyridyl, phenyl, and optionally substituted phenyl;

R4 and R5 are, independently, selected from the group consisting of hydrogen, methyl, (C₂-C₁₂)alkyl, (C₁-C₆)alkenyl, (C₁-C₆)alkyl(C₃-C₆)cycloalkyl, (C₁-C₆)alkyl-phenyl, (C₁-C₆)alkyl-OH, (C₁-C₆)alkyl-CN, (C₁-C₆)alkyl-halogen, (C₁-C₆)alkyl-CF₃, (C₁-C₆)alkyl-OCH₃, and (C₁-C₆)alkyl-O—(C₁-C₆)alkyl-OCH₃; provided that both R4 and R5 are not hydrogen;

X⁻ represents an anion associated with the positive charge of the N atom; including but not limited to chloride, bromide, iodide, sulfate, benzene sulfonate, and toluene sulfonate.

Illustrative examples of this invention include:

2-[(3-endo)-9-methyl-9-azabicyclo[3.3.1]non-3-yl]-1,1-di-2-thienylethanol (3-endo)-3-(2-hydroxy-2,2-di-2-thienylethyl)-9,9-dimethyl-9-azoniabicyclo[3.3.1]nonane iodide;
(3-endo)-3-[2-hydroxy-2,2-bis(2-methylphenyl)ethyl]-9,9-dimethyl-9-azoniabicyclo[3.3.1]nonane iodide;
2-[(3-endo)-9-methyl-9-azabicyclo[3.3.1]non-3-yl]-1,1-bis(2-methylphenyl)ethanol 1,1-dicyclohexyl-2-[(3-endo)-9-methyl-9-azabicyclo[3.3.1]non-3-yl]ethanol 1,1-dicyclopentyl-2-[(3-endo)-9-methyl-9-azabicyclo[3.3.1]non-3-yl]ethanol (3-endo)-3-(2-cyano-2,2-di-2-thienylethyl)-9,9-dimethyl-9-azoniabicyclo[3.3.1]nonane bromide;
(3-endo)-3-(2,2-dicyclopentyl-2-hydroxyethyl)-9,9-dimethyl-9-azoniabicyclo[3.3.1]nonane iodide;
(3-endo)-3-(2,2-dicyclohexyl-2-hydroxyethyl)-9,9-dimethyl-9-azoniabicyclo[3.3.1]nonane iodide;
(3-endo)-3-(2-hydroxy-2,2-di-3-thienylethyl)-9,9-dimethyl-9-azoniabicyclo[3.3.1]nonane bromide;
(3-endo)-3-(2-hydroxy-2,2-diphenylethyl)-9,9-dimethyl-9-azoniabicyclo[3.3.1]nonane bromide;
(3-endo)-3-(2-cyano-2,2-diphenylethyl)-9,9-dimethyl-9-azoniabicyclo[3.3.1.]nonane bromide;
(3-endo)-3-(2,2-diphenylethyl)-9,9-dimethyl-9-azoniabicyclo[3.3.1]nonane bromide;
(3-endo)-3-[2-cyano-2,2-bis(5-methyl-2-thienyl)ethyl]-9,9-dimethyl-9-azoniabicyclo[3.3.1]nonane bromide; and
(3-endo)-3-{2-hydroxy-2,2-bis[2-(methyloxy)phenyl]ethyl}-9,9-dimethyl-9-azoniabicyclo[3.3.1]nonane bromide.

### Methods of Preparation

The compounds of Formula (I) may be obtained by applying synthetic procedures, some of which are illustrated in the Schemes below. The synthesis provided for these Schemes is applicable for producing compounds of Formula (I) having a variety of different R_{X} groups (X=2, 3) which are reacted, employing substituents which are suitably protected, to achieve compatibility with the reactions outlined herein. While the Schemes are shown with compounds only of Formula (I), this is merely for illustration purpose only. 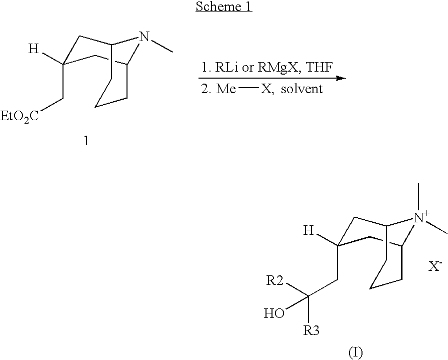
As outlined in Scheme 1, the desired compounds of Formula (I) can be prepared via the reaction of the ester 1 with an excess of organolithium or Grignard reagent. Reaction of the tertiary nitrogen with either methyl iodide or methyl bromide gives the compound of Formula (I).

The required [3.3.1]bicyclic ester 1 can be prepared from pseudopelletierine (2), which is commercially available as the hydrochloride salt. As shown in Scheme 2, the Horner-Emmons reaction of 2 using diethyl(cyanomethyl)phosphonate and sodium hydride provides the alkene 3. Hydrogenation of 3 produced the nitrile 4, which was then hydrolyzed and esterified in situ to give the ester 1. 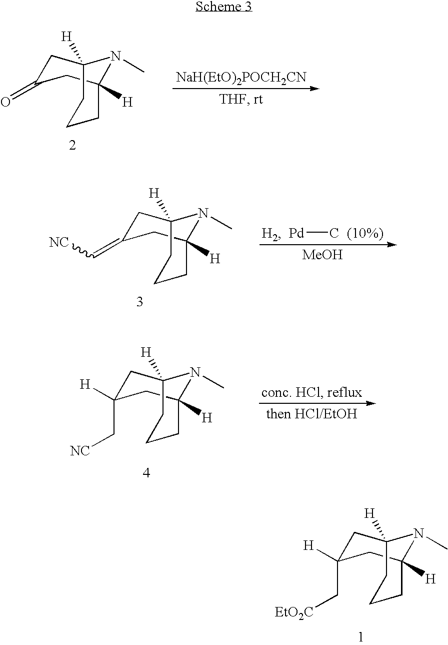
Alternatively, the exo isomer of ester 1 can also be prepared as outlined in Scheme 3. Specifically, a dissolving metal reduction of the alkene 3 with magnesium in MeOH provides the exo orientation of the side chain. The nitrile 5 is then hydrolyzed and esterified as shown to give the exo ester 6. Following a reaction sequence similar to that shown in Scheme 1, the ester 6 is then reacted further to provide the compounds of Formula (I) with an exo side chain. 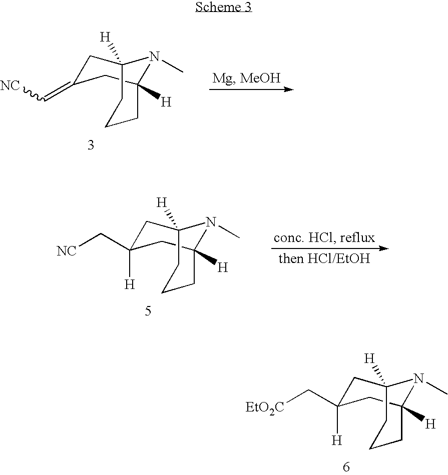
The tertiary alcohols shown in Scheme 1 may be transformed into a tertiary nitrile via a one-pot procedure. Specifically, sequential treatment of the tertiary amine 7 with AlCl₃ and then trimethylsilyl cyanide (TMSCN) gives the compound of Formula (I) where Y═CN, which may then be reacted with methyl bromide to provide the corresponding quaternary amine salt. 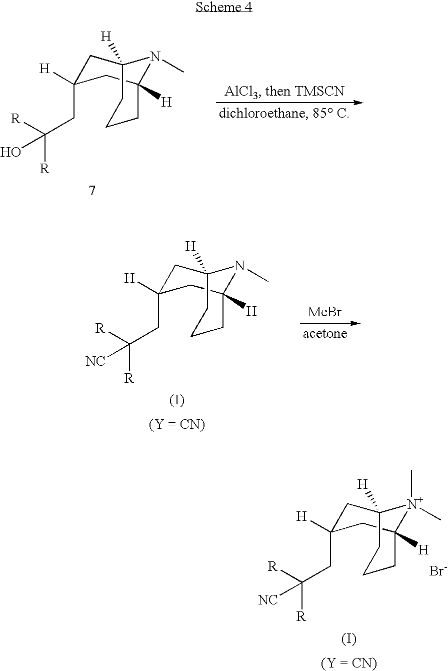
To obtain compounds of Formula (I) where R1=H, the alcohol 7 is dehydrated under acidic conditions to provide the alkene 8 (Scheme 5). Compound 8 is then hydrogenated to give (I) where Y═H, which may then be treated with either methyl bromide or methyl iodide to give the quaternary nitrogen salt.

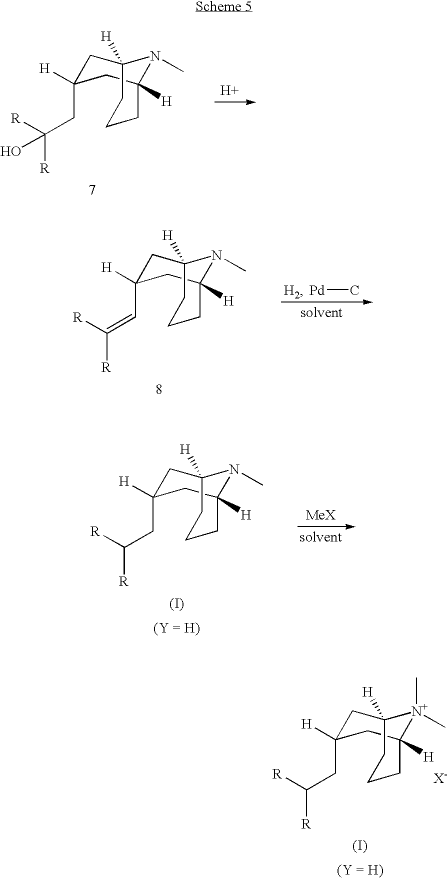

### SYNTHETIC EXAMPLES

The invention will now be described by reference to the following Examples that are merely illustrative and are not to be construed as a limitation of the scope of the present invention. Unless otherwise indicated, all starting materials were obtained from commercial suppliers and used without further purification. All temperatures are given in ° C. Anhydrous solvents were purchased from Aldrich. Thin layer chromatography (t.l.c.) was carried out on silica. Flash chromatography was conducted according to the Still protocol (Still, W. C., et al J. Org. Chem. 1978, 43, 2923-2925) using EMD (Merck) 9385 40-63d silica gel (230-400 mesh) with the indicated solvents unless stated otherwise. All ¹H NMR spectra were taken on a 400 MHz instrument. Analytical LC/MS was conducted under the following conditions:
Liquid Chromatograph System: Shimadzu LC system with SCL-10A Controller and dual UV detector
Autosampler: Leap CTC with a Valco six port injector
Column: 1 mm×40 mm, Aquasil (C18)
Flow Rate: 0.3 mL/min
Injection Volume: 2 μl
Temp: room temperature
Solvents:
   A: 0.02% Trifluoroacetic Acid/Water.

B: 0.018% Trifluoroacetic Acid/Acetonitrile.

Gradient (Linear):

| Time (min) | Duration (min) | A % | B % |
|---|---|---|---|
| 0.00 | 0.00 | 95 | 5 |
| 0.00 | 0.01 | 95 | 5 |
| 0.01 | 3.20 | 10 | 90 |
| 3.21 | 1.00 | 10 | 90 |
| 4.21 | 0.01 | 95 | 5 |
| 4.31 | 0.40 | 95 | 5 |

The Gilson preparatory HPLC was conducted under the following conditions:

Column: 75×33 mm I. D., S-5 μm, 12 nm

Flow rate: 30 mL/min

Injection Volume: 0.800 mL

Room temperature

Solvent A: 0.1% trifluoroacetic acid in water

Solvent B: 0.1% trifluoroacetic acid in acetonitrile

### (±)-(9-Methyl-9-azabicyclo[3.3.1]non-3-ylidene)ethanenitrile (3)

Diethyl(cyanomethyl)phosphonate (5.12 mL, 31.7 mmol) was added dropwise over 6 min to a stirred slurry of 95% NaH (800 mg, 31.7 mmol) in anhydrous THF (32 mL) under argon at room temperature. After stirring for 40 min, a solution of 2 (970 mg, 6.33 mmol) in THF (10 mL) was added in one portion. Stirring was continued for 70 h, whereupon MeOH (5 mL) was added in one portion. The mixture was concentrated under reduced pressure, and the residue was taken up in a 1:1 mixture of H₂O/EtOAc (20 mL). The layers were separated, and the aqueous layer was extracted with EtOAc (4×5 mL). The combined organic layers were dried (Na₂SO₄) and concentrated under reduced pressure. The crude product was purified on a Biotage 40+M cartridge (100 g silica gel) eluting with 3% MeOH/CH₂Cl₂ (2 L), followed by 10% MeOH/CH₂Cl₂ (1 L) at 40 psi to give 886 mg (˜79%) of 3 as a yellow oil. ¹H NMR (CDCl₃) of 3 showed some contaminants between 3.7 and 4.3 ppm, but the material was of suitable purity to carry on for the next step in the reaction sequence.

LC/MS ESI R_{T} 1.13 min MH⁺ 177

### [(3-Endo)-9-methyl-9-azabicyclo[3.3.1]non-3-yl]acetonitrile (4)

Acetyl chloride (0.57 mL, 7.49 mmol) was added dropwise with stirring to MeOH (3.2 mL; caution: exotherm) at room temperature. Compound 3 (880 mg, 4.99 mmol) was dissolved in this solution and then concentrated under reduced pressure. 10% Pd—C (266 mg, 0.25 mmol) was added, and the reaction flask was purged with argon. MeOH (10 mL) was then added, and the flask was purged for 15 min with a H₂ balloon (note: to ensure efficient purging, the H₂ was introduced to the reaction flask via a 4 inch needle, where the needle tip was situated just above the reaction mixture). The reaction was stirred at room temperature for 22 h, whereupon the reaction mixture was filtered through a pad of Celite 521. The filter cake was rinsed with MeOH (3×10 mL), and the combined filtrate was concentrated under reduced pressure. Saturated K₂CO₃ (10 mL) and EtOAc (10 mL) were added, the layers were separated, and the aqueous layer was extracted with EtOAc (3×10 mL). The combined organic layers were dried (Na₂SO₄) and concentrated under reduced pressure. The crude product was purified on a Biotage 25+M cartridge (40 g silica gel) eluting with 5% MeOH/CH₂Cl₂ (500 mL), followed by 10% MeOH/CH₂Cl₂ (1.5 L) at 40 psi to give 517 mg (58%) of 4.

LC/MS ESI R_{T} 0.74 min MH⁺ 179

### Ethyl[(3-endo)-9-methyl-9-azabicyclo[3.3.1]non-3-yl]acetate (1)

A solution of 4 (515 mg, 2.89 mmol) in concentrated HCl (10 mL) was heated at reflux for 2 h and then concentrated under reduced pressure. In a separate flask, a solution of 2 M HCl/EtOH (5 mL) was prepared by dropwise addition of acetyl chloride (0.7 mL, 9.8 mmol) to EtOH (4.3 mL) with stirring (caution: exotherm). This solution was then added to the crude product obtained by hydrolysis of 4, and the reaction was stirred at room temperature for 24 h. The reaction mixture was concentrated under reduced pressure, and the residue was taken up in a 2:1 mixture of saturated K₂CO₃/EtOAc (15 mL). The layers were separated, and the aqueous layer was extracted with EtOAc (3×5 mL). The combined organic layers were dried (Na₂SO₄) and concentrated under reduced pressure to give 516 mg (79%) of 1. Compound 1 was determined to be sufficient by ¹H NMR (CDCl₃) and LC/MS to carry on for the next step in the reaction sequence.

LC/MS ESI R_{T} 1.17 min MH⁺ 226.2

### [(3-Exo)-9-methyl-9-azabicyclo[3.3.1]non-3-yl]acetonitrile (5)

Mg(0) (11 g, 454 mmol) was added to a solution of 3 (2 g, 11.35 mmol) in MeOH (110 mL) under argon. The reaction was stirred at room temperature for 10 min, was cooled to 0° C. (bath temp), and was allowed to warm slowly to room temperature. After 17 h, the reaction (a solid mass) was cooled to 0° C. (bath temp), and concentrated HCl (100 mL) was added portion-wise (exotherm). The reaction was stirred at room temperature for 1 h, and the solids were filtered off. The filter cake was rinsed with EtOAc (2×100 mL, then 2×150 mL). The combined filtrate was dried (Na₂SO₄) and concentrated under reduced pressure. The crude product (690 mg) was purified by flash chromatography on neutral alumina (40 g; Aldrich, 60 Å) eluting with the following sequence of solvents (200 mL each) EtOAc, 0.1% MeOH/EtOAc, 0.2% MeOH/EtOAc, 0.3% MeOH/EtOAc, 0.5% MeOH/EtOAc, 0.75% MeOH/EtOAc, 1% MeOH/EtOAc, 3% MeOH/EtOAc, 5% MeOH/EtOAc. The fractions which were contained 5 (as determined by TLC) were pooled, concentrated under reduced pressure, and taken up in a 1:1 mixture of 6 M NaOH/EtOAc (6 mL). The layers were separated and the aqueous layer was extracted with EtOAc (3×2 mL). The combined organic layers were dried (Na₂SO₄), concentrated under reduced pressure, and dried under high vacuum to give 310 mg (15%) of 5.

LC/MS ESI R_{T} 1.11 min MH⁺ 179.2

### Ethyl[(3-exo)-9-methyl-9-azabicyclo[3.3.1]non-3-yl]acetate (6)

A solution of 5 (310 mg, 1.74 mmol) in concentrated HCl (3 mL) was heated at reflux with stirring for 2 h, whereupon EtOH (10 mL) was added. Stirring continued at room temperature for 12 h, and the EtOH was removed under reduced pressure. NaOH (1.6 g, 40 mmol) and H₂O (2 mL) were added, and the mixture was stirred until all NaOH dissolved. The mixture was extracted with EtOAc (1×5 mL). According to LC/MS, this organic layer contained only the intermediate carboxylic acid and none of 6. The aqueous layer was made acidic with concentrated HCl (2 mL), combined with the organic layer, and concentrated under reduced pressure. The residue was taken up in a 2 M solution of HCl/EtOH (5 mL) and stirred at room temperature for 4 d. The reaction was concentrated under reduced pressure, and a mixture of saturated K₂CO₃ (3 mL) and EtOAc (5 mL) was added. The solids were filtered off and the filter cake was rinsed with EtOAc (4×5 mL). The combined filtrate was washed with saturated NaCl (1×2 mL), dried (Na₂SO₄), and concentrated under reduced pressure to give 273 mg (70%) of 6. The purity of 6 as determined by ¹H NMR (CDCl₃) was considered to be sufficient to carry on for the next step in the reaction sequence.

LC/MS ESI R_{T} 1.04 min MH⁺ 226.2

### Example 1

### 2-[(3-Endo)-9-methyl-9-azabicyclo[3.3.1]non-3-yl]-1,1-di-2-thienylethanol

A solution of 1 (273 mg, 1.21 mmol) in THF (3 mL) was added dropwise with stirring to a 1 M solution of 2-thienyllithium in THF (4.8 mL, 4.8 mmol) at −30° C. (bath temp) under argon. The ice bath was removed and stirring was continued for 5 h, whereupon H₂O (3 mL) was added. The layers were separated, and the aqueous layer was extracted with EtOAc (3×2 mL). The combined organic layers were washed with saturated NaCl (1×1 mL), dried (Na₂SO₄), and concentrated under reduced pressure. The crude product was purified by flash chromatography on silica gel (20 g) eluting with 5% MeOH/CH₂Cl₂ (600 mL), followed by 10% MeOH/CH₂Cl₂ (300 mL) to give 145 mg (48%) of Example 1.

LC/MS ESI R_{T} 1.17 min MH⁺ 226.2

### Example 2

### 3-[(3-Endo)-9-methyl-9-azabicyclo[3.3.1]non-3-yl]-2,2-di-2-thienylpropanenitrile

AlCl₃ (176 mg, 1.33 mmol) was added to a slurry of Example 1 (95 mg, 0.27 mmol) in dichloroethane (5.3 mL) in a 2 dram vial. The vial was sealed with a Teflon-lined screwcap and the reaction was stirred at room temperature for 10 min. Trimethylsilyl cyanide (TMSCN, 0.18 mL, 1.33 mmol) was then added, the vial was resealed, and the reaction was stirred at 85° C. (bath temp) for 20 h. The reaction was stirred at room temperature for 10 min, and a further portion of AlCl₃ (176 mg, 1.33 mmol) was added. Stirring continued for 10 min, whereupon another portion of TMSCN (0.18 mL, 1.33 mmol) was added. The reaction was stirred at 85° C. for 40 h, and the reaction was poured into a 2:1 mixture of saturated K₂CO₃/EtOAc (30 mL) with stirring. The black precipitate was filtered off, and the filter cake was rinsed with EtOAc (3×5 mL). The layers of the filtrate were separated, and the aqueous layer was extracted with EtOAc (3×5 mL). The combined organic layers were dried (Na₂SO₄) and concentrated under reduced pressure. The crude product was purified on a Biotage 25+S cartridge (20 g silica gel) at 5 psi eluting with 3% MeOH/CH₂Cl₂ (1 L), followed by 10% MeOH/CH₂Cl₂ (1 L) to give 45 mg (48%) of Example 2.

LC/MS ESI R_{T} 1.69 min MH⁺ 357.2

### (3-Endo)-3-(2,2-di-2-thienylethenyl)-9-methyl-9-azabicyclo[3.3.1]nonane (7)

Oxalic acid (207 mg, 2.30 mmol) was added to a slurry of Example 1 (200 mg, 0.576 mmol) in H₂O (2 mL) in a 2-dram vial. The reaction vial was sealed with a Teflon-lined screwcap, and the reaction was stirred at 100° C. (bath temp) for 1 h. 6 M NaOH (1 mL) was added, and the mixture was extracted with EtOAc (4×2 mL). The combined organic layers were dried (Na₂SO₄) and concentrated under reduced pressure. The crude product was purified on a Biotage 25+S cartridge (20 g silica gel) at 5 psi eluting with 0.25% aqu NH₄OH/10% MeOH/CH₂Cl₂ (500 mL) to give 135 mg (71%) of 7.

LC/MS ESI R_{T} 1.73 min MH⁺ 329.6

### Example 3

### 2-[(3-Endo)-9-methyl-9-azabicyclo[3.3.1]non-3-yl]-1,1-diphenylethanol

A solution of 1 (315 mg, 1.40 mmol) in THF (7 mL) was added to a 1.5 M solution of PhLi in 70:30 cyclohexane/Et₂O (3.73 mL, 5.6 mmol) at −30° C. (bath temp) under argon. The ice bath was removed, and the reaction was stirred for 3 h, whereupon H₂O (5 mL) was added, followed by EtOAc (5 mL). The layers were separated, and the aqueous layer was extracted with EtOAc (4×2 mL). The combined organic layers were washed with saturated NaCl (1×5 mL), dried (Na₂SO₄), and concentrated under reduced pressure. The crude product was purified on a Biotage 25+S cartridge (20 g silica gel) at 5 psi eluting with 0.5% aqu NH₄OH/10% MeOH/CH₂Cl₂ (2 L) to give 295 mg (63%) of Example 3.

LC/MS ESI R_{T} 1.66 min MH⁺ 336.2

### Example 4

### 3-[(3-Endo)-9-methyl-9-azabicyclo[3.3.1]non-3-yl]-2,2-diphenylpropanenitrile

AlCl₃ (158 mg, 1.19 mmol) was added to a slurry of Example 3 (80 mg, 0.24 mmol) in dichloroethane (4.8 mL) in a 2 dram vial. The vial was sealed with a Teflon-lined screwcap and the reaction was stirred at room temperature for 10 min. TMSCN (0.16 mL, 1.19 mmol) was then added, the vial was resealed, and the reaction was stirred at 85° C. (bath temp) for 15 h and then at room temperature for 10 min. A further portion of AlCl₃ (158 mg, 1.19 mmol) was added, and stirring continued for 10 min, whereupon another portion of TMSCN (0.16 mL, 1.19 mmol) was added. The reaction was stirred at 85° C. for 20 h and then at room temperature for 40 h. A further portion of AlCl₃ (158 mg, 1.19 mmol) and TMSCN (0.16 mL, 1.19 mmol) was added as shown above, and the reaction was stirred at 85° C. for 24 h. The reaction was poured into a 2:1 mixture of saturated K₂CO₃/EtOAc (30 mL) with stirring. The black precipitate was filtered off, and the filter cake was rinsed with EtOAc (3×10 mL). The layers of the filtrate were separated, and the aqueous layer was extracted with EtOAc (3×5 mL). The combined organic layers were dried (Na₂SO₄) and concentrated under reduced pressure. The crude product was purified on a Biotage 25+S cartridge (20 g silica gel) at 5 psi eluting with 5% MeOH/CH₂Cl₂ (500 mL), followed by 10% MeOH/CH₂Cl₂ (500 mL) to give 52 mg (63%) of Example 4.

LC/MS ESI R_{T} 1.82 min MH⁺ 345.2

### (3-Endo)-3-(2,2-diphenylethenyl)-9-methyl-9-azabicyclo[3.3.1]nonane (8)

A solution of Example 3 (208 mg, 0.62 mmol) in conc. HCl (2 mL) in a 2-dram vial sealed with a Teflon-lined screwcap was heated at 110° C. (bath temp) with stirring for 1 h. NaOH (1.2 g) was added portionwise (caution: exotherm), and the mixture was extracted with EtOAc (4×2 mL). The combined organic layers were dried (Na₂SO₄) and concentrated under reduced pressure. The crude product was purified on a Biotage 25+S cartridge (20 g silica gel) eluting with 0.25% aqu NH₄O/10% MeOH/CH₂Cl₂ (500 mL) to give 190 mg (96%) of 8.

LC/MS ESI R_{T} 1.70 min MH⁺ 318.0

### Example 5

### (3-Endo)-3-(2,2-diphenylethyl)-9-methyl-9-azabicyclo[3.3.1]nonane

MeOH (10 mL) was added to a mixture of 8 (85 mg, 0.268 mmol) and 10% Pd—C (14 mg, 0.0134 mmol) under argon, and the flask was purged for 15 min with a H₂ balloon (note: to ensure efficient purging, the H₂ was introduced to the reaction flask via a 4 inch needle, where the needle tip was situated just above the reaction mixture). The reaction was stirred at room temperature for 20 h, whereupon the reaction mixture was filtered through a pad of Celite 521. The filter cake was rinsed with MeOH (4×5 mL), and the combined filtrate was concentrated under reduced pressure to give 85 mg (100%) of Example 5. The purity of Example 5 as determined by ¹H NMR (CDCl₃) and LC/MS was considered to be sufficient to carry on for the next step in the reaction sequence.

LC/MS ESI R_{T} 1.91 min MH⁺ 320.2

### Example 6

### 2-[3-(Endo)-9-Methyl-9-aza-bicyclo[3.3.1]non-3-yl]-1,1-di-thiophen-3-yl-ethanol

3-Bromothiophene (0.303 g, 1.86 mmol) was dissolved in THF (4 mL) and cooled to −78° C. A 2.5 M solution of n-butyl lithium in hexanes (0.78 mL, 1.95 mmol) was added to the solution and stirred at −78° C. for 1 hour. A solution of 1 (0.200 g, 0.887 mmol) was added in THF (4 mL) and stirred at room temperature for 18 hours. TLC of the reaction (1.8% NH₄OH/8% MeOH/90% CH₂Cl₂) showed mostly starting material with some expected product. A solution of 3-lithiothiophene was prepared in a separate flask with 3-bromothiophene (2.89 g, 17.7 mmol) and a 2.5 M solution of n-butyl lithium in hexanes (7 mL). The 3-lithiothiophene solution was transferred at −78° C. via cannula to the reaction mixture and stirred at room temperature for five hours. TLC of the reaction showed a reduction of compound 1. The reaction was quenched with saturated NH₄Cl (30 mL), the THF was concentrated, and ethyl acetate was added (200 mL). The organic layer was washed sequentially with H₂O (150 mL) and saturated NaCl (150 mL), dried (MgSO₄), and concentrated. The crude material was purified by reversed phase HPLC 10-90, 15 min., no TFA, to give 0.020 g (6%) of compound Example 6.

LC/MS ESI R_{T} 1.5 min MH⁺ 348

### Example 7

### 2-[(3-Endo)-9-methyl-9-aza-bicyclo[3.3.1]non-3-yl)]-1,1-bis-(5-methyl-thiophen-2-yl)-ethanol

2-Bromo-5-methyl thiophene (3.14 g, 17.7 mmol) was dissolved in THF (10 mL) and cooled to −78° C. A 2.5 M solution of n-butyl lithium in hexanes (7.25 mL, 17.7 mmol) was added and stirred for 45 min at −78° C. A solution of 1 (0.210 g, 0.93 mmol) in THF (5 mL) was added and stirred at room temperature for 72 h. The reaction was then quenched with H₂O (150 mL) and concentrated. The aqueous layer was extracted with ethyl acetate (3×150 mL). The combined organics were washed with H₂O (150 mL), saturated NaCl (150 mL), and dried (MgSO₄). The crude product was purified on silica gel using the Isco Combiflash with a 40 g prepacked column and a solvent system consisting of 1.8% NH₄OH/8% MeOH/90% CH₂Cl₂, to give 0.172 g (49%) of Example 7.

LC/MS ESI R_{T} 1.74 min MH⁺ 376.

### Example 8

### 3-[(3-Endo)-9-methyl-9-aza-bicyclo[3.3.1]non-3-yl)]2,2-bis-(5-methyl-thiophen-2-yl)-propionitrile

Example 7 (0.100 g, 0.266 mmol) was dissolved in a 10 mL vial in dichloroethane (10 mL) and flushed with argon. To this solution was added AlCl₃ (0.352 g, 2.66 mmol), and the reaction was stirred for 5 minutes. Trimethylsilyl cyanide (0.355 mL, 2.66 mmol) was added and the sealed vial was heated to 85° C. for 18 hours. Additional AlCl₃ (0.352 g, 2.66 mmol) and trimethylsilyl cyanide (0.355 mL, 2.66 mmol) were added and continued heating for 24 hours. Trimethylsilyl cyanide (0.300 mL, 2.25 mmol) was added and stirring continued for 5 hours. The reaction mixture was then cooled to room temperature and poured into a mixture of 15 mL of saturated K₂CO₃/10 mL ethyl acetate, stirred for 15 minutes, and then filtered through Celite 545. The filtrate was extracted with ethyl acetate (3×). The combined organics were washed with H₂O (150 mL), saturated NaCl (150 mL), dried (MgSO₄), and concentrated. The crude product was purified on silica gel using the Isco Combiflash with a 10 g prepacked column and a solvent system consisting of 1.8% NH₄OH/8% MeOH/90% CH₂Cl₂, to give 0.030 g (29%) of Example 8. LC/MS ESI R_{T} 2.01 min MH⁺ 385.

### Example 9

### 1,1-Bis-(2-methoxy-phenyl)-2-[(3-endo)-9-methyl-9-aza-bicyclo[3.3.1]non-3-yl)-ethanol

A 1 M solution of 2-Methoxyphenylmagnesium bromide in THF (0.048 mL, 48.0 mmol) was cooled to 0° C. under argon. Compound 1 (1.1 g, 4.88 mmol) was added as a solution in THF (40 mL) and the reaction was heated at reflux for 18 h. The reaction was then cooled and quenched with saturated NH₄Cl (150 mL). The THF was concentrated and the aqueous layer was extracted with CH₂Cl₂ (3×200 mL). The combined organics were washed with H₂O (150 mL), saturated NaCl (150 mL) and dried with an International Sorbent Technology Phase Separator cartridge (70 mL). The crude product was purified on silica gel using the Isco Combiflash with a 120 g prepacked column and a solvent system consisting of 1.8% NH₄OH/8% MeOH/90% CH₂Cl₂, to give 1.39 g of a mixture of compounds including Example 9.

LC/MS ESI R_{T} 1.74 min MH⁺ 400

### Example 10

### 2-[(3-Endo)-9-methyl-9-azabicyclo[3.3.1]non-3-yl)-1,1-bis(2-methylphenyl)ethanol

To a solution of 1 (300 mg, 1.33 mmol) in THF (15 mL) was added a 2 M solution of 2-methylphenylmagnesium bromide in THF (4 mL, 7.9646 mmol). The reaction mixture was heated at 70° C. for 2 h. Water (5 mL) was added to quench the reaction. The reaction mixture was extracted with ethyl acetate (3×10 mL), dried (MgSO₄), and concentrated. Example 10 (130 mg, 27%) was obtained after purification with combiflash eluting with 9:1 MeOH/CH₂Cl₂.

LC/MS: 2.00 min, M+H, 364.8

### Example 11

### 1,1-Dicyclohexyl-2-[(3-endo)-9-methyl-9-azabicyclo[3.3.1]non-3-yl)ethanol

To a solution of 1 (600 mg, 2.65 mmol) in THF (30 mL) was added 2 M solution of cyclohexylmagnesium bromide in THF (5.3 mL, 10.6 mmol). The reaction mixture was refluxed at 60° C. for 2 h. A solution of saturated aqueous ammonium chloride (10 mL) was added to quench the reaction. The reaction mixture was extracted with ethyl acetate (3×50 mL), dried (MgSO₄) and concentrated. Example 11 (140 mg, 15%) was obtained after purification with combiflash eluting with 9:1 MeOH/CH₂Cl₂.

LC/MS: 2.19 min, M+H, 348.2

### Example 12

### 1,1-Dicyclopentyl-2-[(3-endo)-9-methyl-9-azabicyclo[3.3.1]non-3-yl)ethanol

To a solution of 1 (500 mg, 2.21 mmol) in THF (30 mL) was added a 2 M solution of cyclopentylmagnesium bromide in Et₂O (5.3 mL, 10.62 mmol). The reaction mixture was heated at 60° C. for 3 h. A solution of saturated aqueous ammonium chloride (20 mL) was added to quench the reaction. The reaction mixture was extracted with ethyl acetate (3×50 mL), dried (MgSO₄) and concentrated. Example 12 (150 mg, 19%) was obtained after purification with combiflush eluting with 9:1 MeOH/CH₂Cl₂.

LC/MS: 1.95 min, M+H, 320.2

### Example 13

### 2-[(3-Exo)-9-methyl-9-azabicyclo[3.3.1]non-3-yl]-1,1-di-2-thienylethanol

A solution of 6 (273 mg, 1.21 mmol) in THF (3 mL) was added dropwise with stirring to a 1 M solution of 2-thienyllithium in THF (4.8 mL, 4.8 mmol) at −30° C. (bath temp) under argon. The ice bath was removed and stirring continued at room temperature for 5 h, whereupon H₂O (3 mL) was added. The layers were separated and the aqueous layer was extracted with EtOAc (3×2 mL). The combined organic layers were washed with saturated NaCl (1×1 mL), dried (Na₂SO₄), and concentrated under reduced pressure. The crude product was purified by flash chromatography on silica gel (16 g) eluting with 0.5% aqu NH₄OH/10% MeOH/CH₂Cl₂ (200 mL), followed by 1% aqu NH₄OH/10% MeOH/CH₂Cl₂ (200 mL) and 1.5% aqu NH₄OH/10% MeOH/CH₂Cl₂ to give 323 mg (77%) of Example 13.

LC/MS ESI R_{T} 1.55 min MH⁺ 348.2

### Example 14

### (3-Endo)-3-(2-hydroxy-2,2-di-2-thienylethyl)-9,9-dimethyl-9-azoniabicyclo[3.3.1]nonane iodide

MeI (0.034 mL, 0.547 mmol) was added to a solution of Example 1 (19 mg, 0.0547 mmol) in acetone (1 mL). The reaction was stirred at room temperature for 20 h, and the solvent was removed under reduced pressure. The crude product was transferred to a medium-porosity glass fritted funnel and rinsed with Et₂O (5×1 Example 14.

LC/MS ESI R_{T} 1.70 min MH⁺ 362.2

### Example 15

### (3-Endo)-3-(2-cyano-2,2-di-2-thienylethyl)-9,9-dimethyl-9-azoniabicyclo[3.3.1]nonane bromide

A 2 M solution of MeBr in tert-Butyl methyl ether (1.26 mL, 2.52 mmol) was added to a solution of Example 2 (45 mg, 0.126 mmol) in acetone (1 mL). The reaction was stirred at room temperature for 4.5 days, and the solvent was removed under reduced pressure. The crude product was transferred to a medium-porosity glass fritted funnel and rinsed with Et₂O (4×1 mL), CH₃CN (1×0.5 mL), and CHCl₃ (1×1 mL). The filter cake was dried under high vacuum to give 14 mg (25%) of Example 15.

LC/MS ESI R_{T} 1.76 min MH⁺ 371.2

### Example 16

### (3-Endo)-3-(2-hydroxy-2,2-diphenylethyl)-9,9-dimethyl-9-azoniabicyclo[3.3.1]nonane bromide

A 2 M solution of MeBr in tert-Butyl methyl ether (1.19 mL, 2.38 mmol) was added to a solution of Example 3 (40 mg, 0.119 mmol) in acetone (1 mL). The reaction was stirred at room temperature for 43 h. The precipitate was filtered off, rinsed with Et₂O (1×1 mL), and dried under high vacuum to give 43.5 mg (85%) of Example 16.

LC/MS ESI R_{T} 1.62 min M⁺ 350.0

### Example 17

### (3-Endo)-3-(2-cyano-2,2-diphenylethyl)-9,9-dimethyl-9-azoniabicyclo[3.3.1]nonane bromide

A 2 M solution of MeBr in tert-Butyl methyl ether (1.48 mL, 2.96 mmol) was added to a solution of Example 4 (51 mg, 0.148 mmol) in acetone (1 mL). The reaction was stirred at room temperature for 80 h. The precipitate was filtered off, rinsed with Et₂O (3×1 mL), and dried under high vacuum to give 44 mg (68%) of Example 17.

LC/MS ESI R_{T} 1.78 min MH⁺ 359.0

### Example 18

### (3-Endo)-3-(2,2-diphenylethyl)-9,9-dimethyl-9-azoniabicyclo[3.3.1]nonane bromide

A 2 M solution of MeBr in tert-Butyl methyl ether (0.67 mL, 1.33 mmol) was added to a solution of Example 5 (85 mg, 0.266 mmol) in acetone (1 mL). The reaction was stirred at room temperature for 38 h. The precipitate was filtered off, rinsed with Et₂O (3×1 mL), and dried under high vacuum to give 69 mg (63%) of Example 18.

LC/MS ESI R_{T} 1.85 min MH⁺ 335.4

### Example 19

### (3-Exo)-3-(2-hydroxy-2,2-di-2-thienylethyl)-9,9-dimethyl-9-azoniabicyclo[3.3.1]nonane bromide

A 2 M solution of MeBr in tert-Butyl methyl ether (0.95 mL, 1.9 mmol) was added to a solution of Example 13 (33 mg, 0.095 mmol) in acetone (2 mL). The reaction was stirred at room temperature for 14 h. The reaction was concentrated under reduced pressure, and the residue was triturated with Et₂O (2×5 mL). The washings were filtered, and the combined solid residue was dried under high vacuum to give 37 mg (88%) of Example 19.

LC/MS ESI R_{T} 1.57 min MH⁺ 362.4

### Example 20

### (3-Endo)-3-(2-hydroxy-2,2-di-thiophen-3-yl-ethyl)-9,9-dimethyl-9-azonia-bicyclo[3.3.1]nonane bromide

Example 6 (0.020 g, 0.058 mmol) was dissolved in 1:1 CH₃CN/CHCl₃ (4 mL). A 2 M solution of methyl bromide in t-butyl methyl ether (0.15 mL, 0.29 mmol) was added to the solution. The reaction was heated to 40° C. for 96 hours to give compound 3. The crude product was purified on reversed phase HPLC 10-90, 10 min, no TFA to give 0.005 g (24%) of compound Example 20.

LC/MS ESI R_{T} 1.48 min MH⁺ 362

### Example 21

### (3-Endo)-3-[2-cyano-2,2-bis-(5-methyl-thiophen-2-yl)-ethyl]-9,9-dimethyl-9-azonia-bicyclo[3.3.1]nonane bromide

Example 8 (0.030 g, 0.078 mmol) was dissolved in a 1:1 solution of CH₃CN/CHCl₃ (4 mL). A 2 M solution of methyl bromide in t-butyl methyl ether (0.19 mL, 0.39 mmol) was added to the solution. The reaction was stirred at room temperature for 18 hours to give compound Example 21. The crude product was purified on reversed phase HPLC 10-90, 10 min, no TFA to give 0.004 g (13%) of compound Example 21.

LC/MS ESI R_{T} 1.84 min MH⁺ 400.

### Example 22

### (3-Endo)-3-[2-hydroxy-2,2-bis-(2-methoxy-phenyl)-ethyl]-9,9-dimethyl-9-azonia-bicyclo[3.3.1]nonane bromide

The crude mixture from the preparation of Example 9 (0.20 g, ˜0.506 mmol) was dissolved in a 1:1 solution of CH₃CN/CHCl₃ (6 mL). A 2 M solution of methyl bromide in t-butyl methyl ether (1.27 mL, 2.53 mmol) was added to the solution. The reaction was stirred at room temperature for 72 hours to give compound 8. The crude product was purified on reversed phase HPLC 10-60, 10 min, no TFA to give 0.0584 g (13%) of compound Example 22.

LC/MS ESI R_{T} 1.76 min MH⁺ 411.

### Example 23

### (3-Endo)-3-[2-hydroxy-2,2-bis(2-methylphenyl)ethyl]-9,9-dimethyl-9-azoniabicyclo[3.3.1]nonane iodide

To a solution of 2-[(3-endo)-9-methyl-9-azabicyclo[3.3.1]non-3-yl)-1,1-bis(2-methylphenyl)ethanol (25 mg, 0.068 mmol) in acetone (5 mL) was added methyl iodide (0.5 mL, 8.03 mmol). The reaction mixture was stirred overnight. Example 23 (20 mg, 77%) was obtained after concentration.

LC/MS: 1.99 min, M+: 378.4

### Example 24

### (3-Endo)-3-(2,2-dicyclohexyl-2-hydroxyethyl)-9,9-dimethyl-9-azoniabicyclo[3.3.1]nonane iodide

To a solution of 1,1-dicyclohexyl-2-[(3-endo)-9-methyl-9-azabicyclo[3.3.1]non-3-yl)ethanol (25 mg, 0.072 mmol) in acetone (5 mL) was added methyl iodide (0.5 mL, 8.03 mmol). The reaction mixture was stirred overnight. Example 24 (20 mg, 77%) was obtained after concentration.

LC/MS: 2.23 min, M+: 362.4

### Example 25

### 3-(2,2-dicyclopentyl-2-hydroxyethyl)-9,9-dimethyl-9-azoniabicyclo[3.3.1]nonane iodide

To a solution of 1,1-dicyclopentyl-2-[(3-endo)-9-methyl-9-azabicyclo[3.3.1]non-3-yl)ethanol (40 mg, 0.125 mmol) in acetone (5 mL) was added methyl iodide (0.5 mL, 8.03 mmol). The reaction mixture was stirred for 2 h. Example 25 (31 mg, 82%) was obtained after concentration.

LC/MS: 1.94 min, M+: 334.4

Abbreviations

AlCl₃ Aluminum trichloride

CH₂Cl₂ Dichloromethane

CH₃CN Acetonitrile

ESI Electrospray ionization

Et₂O Diethyl ether

EtOAc Ethyl acetate

HPLC High pressure liquid chromatography

MeBr Methyl bromide

MeI Methyl iodide

TFA Trifluoroacetic acid

THF Tetrahydrofuran

TMSCN Trimethylsilyl cyanide

### BIOLOGICAL EXAMPLES

The inhibitory effects of compounds at the M₃ mAChR of the present invention are determined by the following in vitro and in vivo functional assays:

Analysis of Inhibition of Receptor Activation by Calcium Mobilization:

Stimulation of mAChRs expressed on CHO cells were analyzed by monitoring receptor-activated calcium mobilization as previously described (H. M. Sarau et al, 1999. Mol. Pharmacol. 56, 657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 501 of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels. The emitted fluorescence from all 96 wells is measured simultaneously using a cooled CCD camera. Data points are collected every second. This data was then plotting and analyzed using GraphPad PRISM software.

Muscarinic Receptor Radioligand Binding Assays

Radioligand binding studies using 0.5 nM [³H]-N-methyl scopolamine (NMS) in a SPA format is used to assess binding of muscarinic antagonists to M₁, M₂, M₃, M₄ and M₅ muscarinic acetylcholine receptors. In a 96-well plate, the SPA beads are pre-incubated with receptor-containing membrane for 30 min at 4° C. Then 50 mM HEPES and the test compound are added and incubated at room temperature (shaking) for 2 hours. The beads are then spun down and counted using a scintillation counter.

Evaluation of Potency and Duration of Action in Isolated Guinea Pig Trachea

Tracheae were removed from adult male Hartely guinea pigs (Charles River, Raleigh, N.C.; 400-600 grams) and placed into modified Krebs-Henseleit solution. Composition of the solution was (mM): NaCl 113.0, KCl 4.8, CaCl₂ 2.5, KH₂PO₄ 1.2, MgSO₄ 1.2, NaHCO₃ 25.0 and dextrose 11.0 which was gassed with 95% O₂:5% CO₂ and maintained at 37° C. Each trachea was cleaned of adherent tissue and opened lengthwise. Epithelium was removed by gently rubbing the luminal surface with a cotton-tipped applicator. Individual strips were cut, approximately 2 cartilage rings in width, and suspended via silk suture in 10-ml water-jacketed organ baths containing Krebs-Henseleit solution and connected to Grass FT03C force-displacement transducers. Mechanical responses were recorded isometrically by MP100WS/Acknowledge data acquisition system (BIOPAC Systems, Goleta, Calif., www.biopac.com) run on Apple G4 computers. The tissues were equilibrated under a resting tension of 1.5 g, determined to be optimal by length-tension evaluation, and washed with Krebs-Henseleit solution every 15 minutes for one hour. After the equilibration period pulmonary tissues were contracted with 10 uM carbachol until reaching plateau, which served as a reference contraction for data analysis. Tissues were then rinsed every 15 minutes over 1 hour until reaching baseline tone. The preparations were then left for at least 30 minutes before the start of the experiment.

Concentration-response curves were obtained by a cumulative addition of carbachol in half-log increments (Van Rossum, 1963, Arch. Int. Pharmacodyn., 143:299), initiated at 1 nM. Each concentration was left in contact with the preparation until the response plateaued before the addition of the subsequent carbachol concentration. Paired tissues were exposed to mAChR antagonist compounds or vehicle for 30 min before carbachol cumulative concentration-response curves were generated. All data is given as mean ±standard error of the mean (s.e.m.) with n being the number of different animals.

For superfusion (duration of action) studies, the tissues were continuously superfused with Krebs-Henseleit solution at 2 ml/min for the duration of the experiment. Stock solutions of agonist and antagonist were infused (0.02 ml/min) via 22-gauge needle inserted into the superfusion tubing. Mechanical responses were recorded isometrically using a commercially-available data acquisition system (MP100WS/Acknowledge; BIOPAC Systems, Goleta, Calif., www.biopac.com) interfaced with a Macintosh G4 computer (Apple, Cupertino, Calif. www.apple.com). The tissues were suspended under an optimal resting tension of 1.5 g. After a 60 min equilibration period, the tissues were contracted with carbachol (1 uM) for the duration of the experiment. Upon reaching a sustained contraction isoproterenol (10 uM) was administered to maximally relax the tissue, and this change served as a reference. Isoproterenol exposure was halted and the carbachol-induced tension allowed to recover. Muscarinic receptor antagonists infused at a single concentration per tissue until a sustained level of inhibition was attained. The compound was then removed and, once again, the carbachol-induced tension was allowed to recover.

The following parameters were determined for each concentration of antagonist, and expressed as the mean ±S.E.M. for n individual animals. Inhibition of the carbachol-induced contraction was expressed as a percent of the reference response (isoproterenol) and the time required to reach one-half of this relaxation was measured (onset of response). The tension recovery following removal of the compound was determined as was the time required to reach one-half of the maximum tension recovery (offset of response). At 60 and 180 minutes after removal of the antagonist the remaining level of inhibition was determined and expressed as a percent of the isoproterenol reference.

Antagonist concentration-response curves were obtained by plotting the maximal relaxation data at 0, 60 and 180-min following antagonist withdrawal. Recovery, termed shift, was calculated from the ratio of the O-min inhibition curve IC₅₀ and the concentration of compound yielding a similar tension recovery at 60 and 180 minutes.

Halftimes for onset and offset of response were plotted vs. corresponding concentration and the data were fit with non-linear regression. These values were extrapolated at the IC₅₀ (determined from the inhibition concentration-response curve) and designated Ot₅₀ (time required, at the IC₅₀ concentration, to reach half of the onset response) and Rt50 (time required, at the IC₅₀ concentration, to reach half of the recovery response).

Methacholine-Induced Bronchoconstriction—Potency and Duration of Action

Airway responsiveness to methacholine was determined in awake, unrestrained Balb C mice (n=6 each group). Barometric plethysmography was used to measure enhanced pause (Penh), a unitless measure that has been shown to correlate with the changes in airway resistance that occur during bronchial challenge with methacholine(2). Mice were pre-treated with 50 μl of compound (0.003-10 μg/mouse) in 50 μl of vehicle (10% DMSO) intranasally (i.n.) and were then placed in the plethysmography chamber a given amount of time following drug administration (15 min-96 h). For potency determination, a dose response to a given drug was performed, and all measurements were taken 15 min following i.n. drug administration. For duration of action determination, measurements were taken anywhere from 15 min to 96 hours following i.n. drug administration.

Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software. This experiment allows the determination of duration of activity of the administered compound.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis.

### Formulation

### Administration

Accordingly, the present invention further provides a pharmaceutical formulation comprising a compound of formula (I), or a pharmaceutically acceptable salt, solvate, or physiologically functional derivative (e.g., salts and esters) thereof, and a pharmaceutically acceptable carrier or excipient, and optionally one or more other therapeutic ingredients.

Hereinafter, the term “active ingredient” means a compound of formula (I), or a pharmaceutically acceptable salt, solvate, or physiologically functional derivative thereof.

Compounds of formula (I) will be administered via inhalation via the mouth or nose.

Dry powder compositions for topical delivery to the lung by inhalation may, for example, be presented in capsules and cartridges of for example gelatine, or blisters of for example laminated aluminium foil, for use in an inhaler or insufflator. Powder blend formulations generally contain a powder mix for inhalation of the compound of the invention and a suitable powder base (carrier/diluent/excipient substance) such as mono-, di- or poly-saccharides (e.g., lactose or starch), organic or inorganic salts (e.g., calcium chloride, calcium phosphate or sodium chloride), polyalcohols (e.g., mannitol), or mixtures thereof, alternatively with one or more additional materials, such additives included in the blend formulation to improve chemical and/or physical stability or performance of the formulation, as discussed below, or mixtures thereof. Use of lactose is preferred. Each capsule or cartridge may generally contain between 20 μg-10 mg of the compound of formula (I) optionally in combination with another therapeutically active ingredient. Alternatively, the compound of the invention may be presented without excipients, or may be formed into particles comprising the compound, optionally other therapeutically active materials, and excipient materials, such as by co-precipitation or coating.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant as an inhaler having a reservoir form pack suitable for comprising multiple (un-metered doses) of medicament in dry powder form and including means for metering medicament dose from the reservoir to a delivery position. The metering means may for example comprise a metering cup or perforated plate, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based, pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disk-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Spray compositions for topical delivery to the lung by inhalation may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain the compound of formula (I) optionally in combination with another therapeutically active ingredient and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants eg oleic acid or lecithin and cosolvents eg ethanol. Pressurised formulations will generally be retained in a canister (eg an aluminium canister) closed with a valve (eg a metering valve) and fitted into an actuator provided with a mouthpiece.

Medicaments for administration by inhalation desirably have a controlled particle size. The optimum aerodynamic particle size for inhalation into the bronchial system for localized delivery to the lung is usually 1-10 μm, preferably 2-5 μm. The optimum aerodynamic particle size for inhalation into the alveolar region for achieving systemic delivery to the lung is approximately 0.5-3 μm, preferably 1-3 μm. Particles having an aerodynamic size above 20 μm are generally too large when inhaled to reach the small airways. Average aerodynamic particle size of a formulation may be measured by, for example cascade impaction. Average geometric particle size may be measured, for example by laser diffraction, optical means.

To achieve a desired particle size, the particles of the active ingredient as produced may be size reduced by conventional means eg by controlled crystallization, micronisation or nanomilling. The desired fraction may be separated out by air classification. Alternatively, particles of the desired size may be directly produced, for example by spray drying, controlling the spray drying parameters to generate particles of the desired size range. Preferably, the particles will be crystalline, although amorphous material may also be employed where desirable. When an excipient such as lactose is employed, generally, the particle size of the excipient will be much greater than the inhaled medicament within the present invention, such that the “coarse” carrier is non-respirable. When the excipient is lactose it will typically be present as milled lactose, wherein not more than 85% of lactose particles will have a MMD of 60-90 μm and not less than 15% will have a MMD of less than 15 μm. Additive materials in a dry powder blend in addition to the carrier may be either respirable, i.e., aerodynamically less than 10 microns, or non-respirable, i.e., aerodynamically greater than 10 microns.

Suitable additive materials which may be employed include amino acids, such as leucine; water soluble or water insoluble, natural or synthetic surfactants, such as lecithin (e.g., soya lecithin) and solid state fatty acids (e.g., lauric, palmitic, and stearic acids) and derivatives thereof (such as salts and esters); phosphatidylcholines; sugar esters. Additive materials may also include colorants, taste masking agents (e.g., saccharine), anti-static-agents, lubricants (see, for example, Published PCT Patent Appl. No. WO 87/905213, the teachings of which are incorporated by reference herein), chemical stabilizers, buffers, preservatives, absorption enhancers, and other materials known to those of ordinary skill.

Sustained release coating materials (e.g., stearic acid or polymers, e.g. polyvinyl pyrolidone, polylactic acid) may also be employed on active material or active material containing particles (see, for example, patent Nos. U.S. Pat. No. 3,634,582, GB 1,230,087, GB 1,381,872, the teachings of which are incorporated by reference herein).

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing an effective dose, as herein before recited, or an appropriate fraction thereof, of the active ingredient.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

What is claimed is:

1. A compound according to Formula (I) represented by the structure: 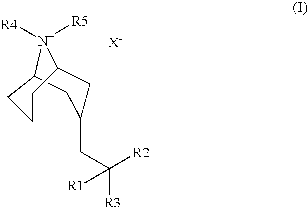
wherein:
the orientation of the alkyl chain attached to the tropane ring is either exo or endo;
R1 is, independently, OH, CN or hydrogen;
R2 and R3 are, independently, selected from the group consisting of straight or branched chain lower alkyl groups, having preferably from 1 to 6 carbon atoms, cycloalkyl groups, having from 5 to 6 carbon atoms, cycloalkyl-alkyl, having 6 to 10 carbon atoms, 2-thienyl, optionally substituted 2-thienyl, 3-thienyl, optionally substituted 3-thienyl, 2-pyridyl, phenyl, and optionally substituted phenyl;
R4 and R5 are, independently, selected from the group consisting of hydrogen, methyl, (C₂-C₁₂)alkyl, (C₁-C₆)alkenyl, (C₁-C₆)alkyl(C₃-C₆)cycloalkyl, (C₁-C₆)alkyl-phenyl, (C₁-C₆)alkyl-OH, (C₁-C₆)alkyl-CN, (C₁-C₆)alkyl-halogen, (C₁-C₆)alkyl-CF₃, (C₁-C₆)alkyl-OCH₃, and (C₁-C₆)alkyl-O—(C₁-C₆)alkyl-OCH₃; provided that both R4 and R5 are not hydrogen; and
X⁻ represents an anion associated with the positive charge of the N atom.

2. A compound according to claim 1 wherein X□is selected from the group consisting of chloride, bromide, iodide, sulfate, benzene sulfonate, and toluene sulfonate.

3. A compound according to claim 1 selected from the group of:
2-[(3-endo)-9-methyl-9-azabicyclo[3.3.1]non-3-yl]-1,1-di-2-thienylethanol (3-endo)-3-(2-hydroxy-2,2-di-2-thienylethyl)-9,9-dimethyl-9-azoniabicyclo[3.3.1]nonane iodide;
(3-endo)-3-[2-hydroxy-2,2-bis(2-methylphenyl)ethyl]-9,9-dimethyl-9-azoniabicyclo[3.3.1]nonane iodide;
2-[(3-endo)-9-methyl-9-azabicyclo[3.3.1]non-3-yl]-1,1-bis(2-methylphenyl)ethanol 1,1-dicyclohexyl-2-[(3-endo)-9-methyl-9-azabicyclo[3.3.1]non-3-yl]ethanol 1,1-dicyclopentyl-2-[(3-endo)-9-methyl-9-azabicyclo[3.3.1]non-3-yl]ethanol (3-endo)-3-(2-cyano-2,2-di-2-thienylethyl)-9,9-dimethyl-9-azoniabicyclo[3.3.1]nonane bromide;
(3-endo)-3-(2,2-dicyclopentyl-2-hydroxyethyl)-9,9-dimethyl-9-azoniabicyclo[3.3.1]nonane iodide;
(3-endo)-3-(2,2-dicyclohexyl-2-hydroxyethyl)-9,9-dimethyl-9-azoniabicyclo[3.3.1]nonane iodide;
(3-endo)-3-(2-hydroxy-2,2-di-3-thienylethyl)-9,9-dimethyl-9-azoniabicyclo[3.3.1]nonane bromide;
(3-endo)-3-(2-hydroxy-2,2-diphenylethyl)-9,9-dimethyl-9-azoniabicyclo[3.3.1]nonane bromide;
(3-endo)-3-(2-cyano-2,2-diphenylethyl)-9,9-dimethyl-9-azoniabicyclo[3.3.1]nonane bromide;
(3-endo)-3-(2,2-diphenylethyl)-9,9-dimethyl-9-azoniabicyclo[3.3.1]nonane bromide;
(3-endo)-3-[2-cyano-2,2-bis(5-methyl-2-thienyl)ethyl]-9,9-dimethyl-9-azoniabicyclo[3.3.1]nonane bromide; and
(3-endo)-3-{2-hydroxy-2,2-bis[2-(methyloxy)phenyl]ethyl}-9,9-dimethyl-9-azoniabicyclo[3.3.1]nonane bromide.

4. A pharmaceutical composition for the treatment of muscarinic acetylcholine receptor mediated diseases comprising a compound according to claim 1 and a pharmaceutically acceptable carrier thereof.

5. A method of inhibiting the binding of acetylcholine to its receptors in a mammal in need thereof comprising administering a safe and effective amount of a compound according to claim 1.

6. A method of treating a muscarinic acetylcholine receptor mediated disease, wherein acetylcholine binds to said receptor, comprising administering a safe and effective amount of a compound according to claim 1.

7. A method according to claim 6 wherein the disease is selected from the group consisting of chronic obstructive lung disease, chronic bronchitis, asthma, chronic respiratory obstruction, pulmonary fibrosis, pulmonary emphysema and allergic rhinitis.

8. A method according to claim 6 wherein administration is via inhalation via the mouth or nose.

9. A method according to claim 6 wherein administration is via a medicament dispenser selected from a reservoir dry powder inhaler, a multi-dose dry powder inhaler or a metered dose inhaler.

10. A method according to claim 9 wherein the compound has a duration of action of 24 hours or more.
